(19) United States
(12) Patent Application Publication
(10) Pub. No.: US 2024/0344125 A1
(43) Pub. Date: Oct. 17, 2024

(54) Nucleotide Sequence Identification Method

(71) Applicant: Hitachi High-Tech Corporation, Minato-ku, Tokyo (JP)

(72) Inventors: Noriyuki SUMIDA, Tokyo (JP); Hirokazu KATO, Tokyo (JP); Michiru FUJIOKA, Tokyo (JP)

(21) Appl. No.: 18/685,199

(22) PCT Filed: Sep. 6, 2021
(86) PCT No.: PCT/JP2021/032625

§ 371 Date: Feb. 20, 2024

(51) Int. Cl. C12Q 1/6874; C12Q 1/6844

(52) U.S. Cl. CPC C12Q 1/6874; C12Q 1/6844

(57) ABSTRACT

Provided is a nucleotide sequence identification method for analyzing a double-stranded DNA in one reaction system while reducing cost and labor. A method for identifying a nucleotide sequence in a target polynucleotide complementary pair constituting a double strand includes: a step of identifying, by using a first primer having a target recognition site configured to form a complementary pair with a part of one target polynucleotide of the target polynucleotide complementary pair, a nucleotide sequence in the one target polynucleotide; and a step of identifying, by using a second primer having a target recognition site configured to form a complementary pair with a part of the other target polynucleotide of the target polynucleotide complementary pair and a reaction stop site configured to stop a nucleotide extension reaction, a nucleotide sequence in the other target polynucleotide, in which a mobility of a second reaction product obtained by the second primer performing extension is smaller than a mobility of a first reaction product obtained by the first primer performing extension.

### TECHNICAL FIELD

The present invention relates to a nucleotide sequence identification method.

### BACKGROUND ART

In a polynucleotide, a molecule in which deoxyribose and deoxyribose are linked in a chain is referred to as a DNA strand. Generally, in a living body, a double-stranded structure is formed in which one DNA strand is intertwined with the other DNA strand, which is configured with a complementary base, in a spiral shape. The complementary bases include thymine (T) for adenine (A) and guanine (G) for cytosine (C). The respective complementary bases are paired via a hydrogen bond. In the present description, one strand is referred to as a top strand and the other is referred to as a bottom strand for convenience. Further, a procedure for obtaining base sequence information on the polynucleotide by analysis is referred to as sequencing.

The base sequence information on the polynucleotide is used for identification of a pathogen, detection of a sudden mutation on a genomic DNA related to cancers, and prediction of drug resistance, effectiveness, and prognosis. In view of an influence on health of a subject, accuracy of the information obtained by the analysis needs to be high.

As described above, when one base in a base pair is determined, the other can be uniquely identified. Therefore, in principle, it is sufficient that the sequencing is performed on only one strand. However, in practice, the accuracy decreases due to various factors such as a device failure and a handling error. Therefore, to increase the accuracy of the sequence information, each strand of a double-stranded DNA may be sequenced.

For example, PTL 1 discloses a method of determining a consensus sequence of nucleotides in an analysis object DNA segment, and the method includes a step of linking ends of a top strand and a bottom strand of the analysis object DNA segment by a covalent bond and a step of sequencing both the top strand and the bottom strand by a sequencing method using a polymerase.

In addition, PTL 2 discloses a method for determining a sequence of one or more nucleotides in a target polynucleotide, in which a target-specific primer contains a target binding segment and a mobility reduction portion that does not bind to the target.

Further, PTL 3 discloses that by using a tail configured with polynucleotides having different lengths, a mobility of a PCR product is reduced, and a plurality of gene loci are analyzed at once.

### CITATION LIST

### Patent Literature

PTL 1: JP2011-515102A
PTL 2: JP2002-536980A
PTL 3: U.S. Pat. No. 6,197,510B

### SUMMARY OF INVENTION

### Technical Problem

In the technique disclosed in PTL 1, a molecule in which one end of a double-stranded DNA is connected by a hairpin loop or a cyclic molecule in which both ends of the double-stranded DNA are connected by a hairpin loop is prepared. However, preparing such molecules requires complicated work of linking of the molecule via the hairpin loop and purification. The techniques disclosed in PTLs 2 and 3 are methods for analyzing one strand of a double-stranded DNA.

An object of the invention is to provide a nucleotide sequence identification method for analyzing a double-stranded DNA in one reaction system while reducing cost and labor.

### Solution to Problem

To solve the technical problem described above, the invention provides a nucleotide sequence identification method for identifying one or more nucleotide sequences in a target polynucleotide complementary pair constituting a double strand, the nucleotide sequence identification method including: a step of identifying, by using a first primer having a target recognition site configured to form a complementary pair by hydrogen bonding with a part of one target polynucleotide of the target polynucleotide complementary pair, one or more nucleotide sequences in the one target polynucleotide; and a step of identifying, by using a second primer having a target recognition site configured to form a complementary pair by hydrogen bonding with a part of the other target polynucleotide of the target polynucleotide complementary pair and a reaction stop site configured to stop a nucleotide extension reaction by a DNA polymerase, one or more nucleotide sequences in the other target polynucleotide, in which a mobility of a second reaction product obtained by the second primer extending a complementary strand on the other target polynucleotide is smaller than a mobility of a first reaction product obtained by the first primer extending a complementary strand on the one target polynucleotide.

### Advantageous Effects of Invention

According to the invention, it is possible to provide a nucleotide sequence identification method for analyzing a double-stranded DNA in one reaction system while reducing cost and labor.

### BRIEF DESCRIPTION OF DRAWINGS

FIG. 1 is a diagram showing a concept of a nucleotide sequence identification method according to Embodiment 1.

FIG. 2 is a flowchart showing a procedure of identifying a nucleotide sequence.

FIG. 3 is a diagram showing information on a primer.

FIG. 4 is a flowchart showing an effect confirmation procedure.

FIG. 5 is a diagram showing temperature conditions in a cycle sequence reaction.

FIG. 6 is a conceptual diagram of a DNA and primers used in Example.

FIG. 7 is electropherograms obtained as a result of electrophoresis for Example and Comparative Examples.

FIG. 8 is a conceptual diagram of a DNA and primers according to Embodiment 4.

### DESCRIPTION OF EMBODIMENTS

In the present description, a DNA strand whose sequence is to be determined is referred to as an analysis object. The analysis object can be prepared as one incorporated into an M13 phage vector or a plasmid. A part of a DNA fragment prepared by a PCR may be the analysis object. An entire DNA strand including the analysis object is referred to as a target polynucleotide. Hereinafter, a method for identifying one or more nucleotide sequences in a target polynucleotide complementary pair constituting a double strand will be described by taking four embodiments as examples.

### Embodiment 1

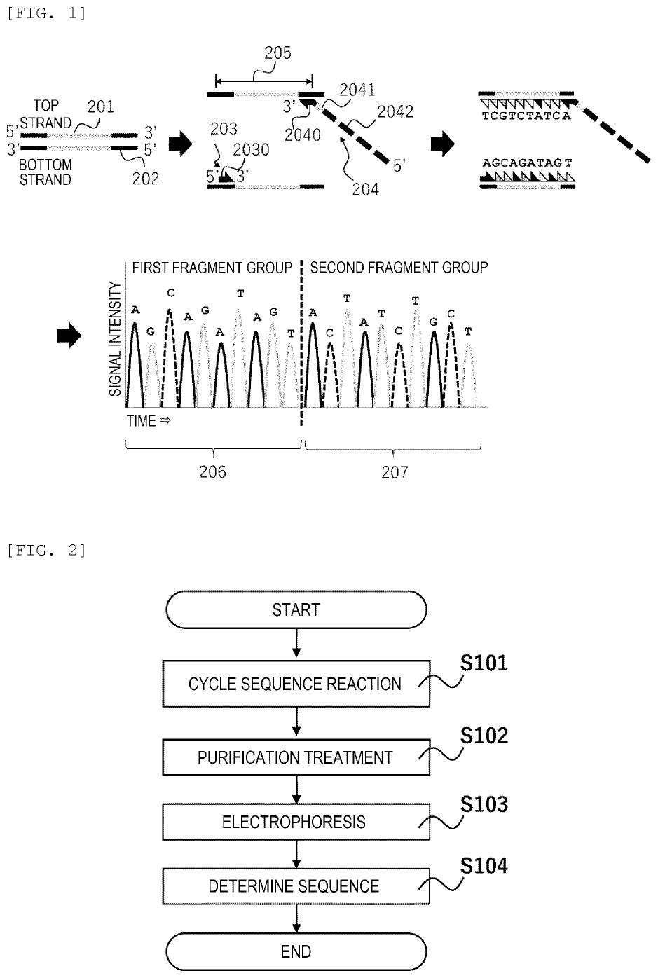

Embodiment 1 will be described with reference to FIGS. 1 to 7. FIG. 1 is a diagram showing a concept of a nucleotide sequence identification method according to Embodiment 1.

A cycle sequence reaction requires at least the following reagents. That is, a target polynucleotide, a deoxyribonucleoside triphosphate for each of four types of bases (adenine (A), guanine (G), cytosine (C), and thymine (T)), a dideoxyribonucleoside triphosphate for each of the four types of bases, a thermostable DNA polymerase, and two types of primers to be described later.

Here, the two types of primers used in the present embodiment will be described. For convenience, one primer is referred to as a first primer 203, and the other primer is referred to as a second primer 204. The first primer 203 and the second primer 204 contain bases complementary to a part of a target polynucleotide 202. As shown in FIG. 1, the first primer 203 and the second primer 204 are designed such that 3′ sides of a DNA face an analysis object 201. Therefore, the first primer 203 and the second primer 204 form a complementary pair with DNA strands different from each other. The complementary bases are usually about 10 to 30 consecutive bases. A nucleotide sequence in one target polynucleotide is identified by using the first primer 203, and a nucleotide sequence in the other target polynucleotide is identified by using the second primer 204.

In the present embodiment, a mobility of a second reaction product obtained by the second primer 204 extending a complementary strand on the other target polynucleotide is smaller than that of a first reaction product obtained by the first primer 203 extending a complementary strand on the one target polynucleotide. Accordingly, since the second reaction product is detected generally later than the first reaction product during electrophoresis, analysis of the double strand can be performed in one reaction system, and efficiency of the analysis can be improved. Hereinafter, a specific configuration of each primer will be described with reference to FIG. 1.

First, the first primer 203 has a target recognition site 2030 that forms the complementary pair by hydrogen bonding with a part of the one target polynucleotide of the target polynucleotide complementary pair.

In contrast, the second primer 204 has a target recognition site 2040 that forms the complementary pair by hydrogen bonding with a part of the other target polynucleotide of the target polynucleotide complementary pair, a reaction stop site 2041, and a mobility reduction site 2042.

The reaction stop site 2041 is located on a 5′ side of the target recognition site 2040 in the second primer 204, and contains a compound for stopping a nucleotide extension reaction by a thermostable DNA polymerase. Typical examples of the compound constituting the reaction stop site 2041 include inosine, a ribonucleoside, an amino acid residue, and a polyethylene glycol. The reaction stop site 2041 is linked to the mobility reduction site 2042.

The mobility reduction site 2042 is located on a 5′ side of the reaction stop site 2041 in the second primer 204, and serves to reduce a moving speed of a reaction product. Here, for convenience, a DNA fragment group extended from a 3′ end of the first primer 203 is referred to as a first fragment group, and a DNA fragment group extended from a 3′ end of the second primer 204 is referred to as a second fragment group. That is, the mobility reduction site 2042 is configured with a compound that reduces a moving speed of the second fragment group in a separation medium in the electrophoresis. More specifically, the mobility reduction site 2042 makes a moving speed of a molecule whose strand length is minimum in the second fragment group slower than a moving speed of a molecule whose strand length is maximum in the first fragment group. As the mobility reduction site 2042, a polynucleotide, an amino acid residue, and a polyethylene glycol can be used.

Next, a length of the second primer 204 will be described by taking an example in which a polynucleotide is used as the mobility reduction site 2042. First, since the second primer 204 is longer than the first primer 203, a mobility of a reaction product derived from the second primer 204 is smaller than a mobility of a reaction product derived from the first primer 203, making it easier to distinguish and detect the reaction products. The mobility of the reaction product derived from the second primer 204 can be adjusted with high accuracy by appropriately setting a length of the mobility reduction site 2042.

In addition, when a region sandwiched by the two primers, specifically, a strand length from a 5′ end of the target recognition site in the first primer 203 to a 5′ end of the target recognition site in the second primer 204 is an amplification region 205 (see FIG. 1), it is desirable that the mobility reduction site 2042 is longer than the amplification region 205. If the mobility reduction site 2042 does not have a strand length equal to or greater than that of the amplification region 205, a peak overlaps in a partial region of the first fragment group and the second fragment group, and therefore it is difficult to determine the peak in the region. On the other hand, when the mobility reduction site 2042 is a polynucleotide having a strand length equal to or greater than that of the amplification region 205, analysis accuracy is improved. Further, when the mobility reduction site 2042 is longer than the amplification region, a gap is generated between the first fragment group and the second fragment group, and therefore there is an advantage that a boundary between the fragment groups is easily determined.

Further, it is desirable that the polynucleotide constituting the mobility reduction site 2042 does not have a region in which bases complementary to the target polynucleotide are consecutive. This is because if there is such a region, the mobility reduction site 2042 starts annealing in the region, which causes noise. To eliminate such a region, it is effective to make thermal stability when the mobility reduction site 2042 forms the complementary pair smaller than thermal stability when the target recognition site 2040 forms the complementary pair.

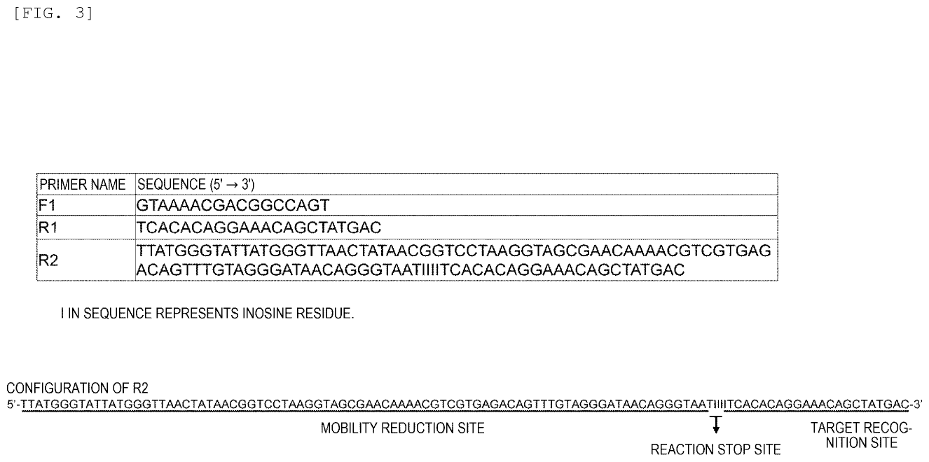

Next, a method for specifying a nucleotide sequence by using the primers described above will be specifically described. FIG. 2 is a flowchart showing a procedure of identifying the nucleotide sequence.

As shown in FIG. 2, first, a cycle sequence reaction shown in step S101 is performed.

Here, a reaction system of the cycle sequence reaction will be described. During the cycle sequence reaction, in addition to the first primer 203, the second primer 204, the target polynucleotide, and the thermostable DNA polymerase, a deoxyribonucleoside triphosphate (dATP, dCTP, dGTP, and dTTP; hereinafter, may be collectively referred to as dNTPs) for each of four types of bases and a dideoxyribonucleoside triphosphate (ddATP, ddCTP, ddGTP, and ddTTP; hereinafter, may be collectively referred to as dNTPs) that is an analog of each dNTP are prepared as substrates. Here, labeled ddNTPs labeled with various fluorescent substances can be used as the ddNTPs. The fluorescent substance is labeled with a fluorescent substance that emits fluorescence having different wavelengths for each of the four types of bases. As a solvent for dissolving a mixed substance necessary for the cycle sequence reaction, a buffer solution capable of adjusting a pH of the reaction system during the cycle sequence reaction to a pH range suitable for polymerase activity of the thermostable DNA polymerase used is used. As the buffer solution, a Tris-HCl buffer, a Tris-acetate buffer, a HEPES-KOH buffer, and a phosphate buffer can be used. The reaction system of the cycle sequence reaction contains metal ions such as Mg2+ and K+ in addition to the target polynucleotide and the mixed substance necessary for the cycle sequence reaction. Further, an SH reducing agent such as 2-mercaptoethanol or dithiothreitol may be appropriately added to improve the polymerase activity. An operator can appropriately adjust concentrations of the target polynucleotide and the mixed substance necessary for the cycle sequence reaction in a solution for performing the cycle sequence reaction. As the mixed substance necessary for the cycle sequence reaction, a commercially available cycle sequence reaction reagent kit can be used. Examples thereof include a BigDye™ terminators v1.1 cycle sequencing kit, a BigDye™ terminator v3.1 cycle sequencing kit, dRhodamine terminator cycle sequencing kits, and dGTP BigDye™ terminator cycle sequencing kits from Applied Biosystems™.

Next, each step in the cycle sequence reaction will be described. Compounds necessary for the above-described cycle sequence reaction are mixed. Regarding the primer, as described above, two types of primers are used. In a cycle sequence, a temperature cycle including a step of converting a double-stranded DNA into a single strand (hereinafter referred to as a denaturing step), a step of forming a complementary pair by hydrogen bonding a part of complementary regions of the target polynucleotide and the primer (hereinafter referred to as an annealing step), and a step of extending a complementary strand by adding a dNTP or a ddNTP to the 3′ end of each primer with the thermostable DNA polymerase (hereinafter referred to as an extending step) is repeated about 25 to 40 times. Generally, the denaturing step is performed at 96° C. for 10 seconds, the annealing step is performed at 50° C. for 5 seconds, and the extending step is performed at 60° C. for 4 minutes. In the first denaturing step (preheating), a long time of about 1 minute to 10 minutes can be set to sufficiently denature a template DNA.

According to the present cycle sequence reaction, a DNA fragment group complementary to a DNA whose base sequence is to be determined and having a different strand length is synthesized as the reaction product. A strand length of the first fragment group derived from the first primer 203 is generally smaller than a strand length of the second fragment group derived from the second primer 204.

After the cycle sequence reaction, a purification treatment (step S102) is performed. An object of the purification treatment is to exchange a solvent suitable for the electrophoresis and to remove the unreacted dNTP, ddNTP, and primers. As a purification method, an ethanol precipitation method, gel filtration, or the like can be appropriately selected and used by the operator. A commercially available DNA purification kit can also be used.

The purified reaction product is separated and detected by the electrophoresis (step S103). The DNA fragment group is separated by a molecular screening effect of the separation medium, and a fluorescence signal from a labeled substance derived from the labeled ddNTPs is detected. Thereafter, a base sequence is determined based on the detected signal (step S104). A type of the electrophoresis method is not particularly limited. In addition to electrophoresis using a modified polyacrylamide gel, which can separate differences in a single base, a capillary electrophoresis device can be used. Hereinafter, a case in which a capillary electrophoresis device is used will be described as an example.

In the present embodiment, a moving speed of the first fragment group is generally larger than that of the second fragment group. Therefore, as shown in FIG. 1, a signal 206 derived from the first fragment group is generally detected earlier than a signal 207 derived from the second fragment group. As a result, the sequence information on the double-stranded DNA can be obtained by one cycle sequence reaction and electrophoresis. Further, in the present embodiment, since the fluorescent substance having a different wavelength is used for each of the four types of bases, the fluorescence signal can be detected by one capillary, which leads to improvement in workability and processing capability.

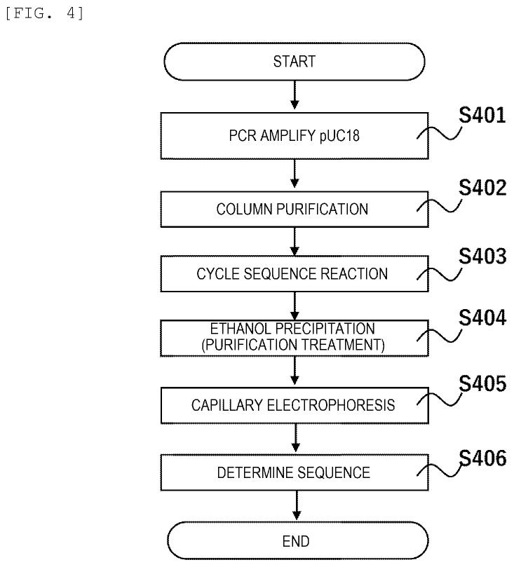

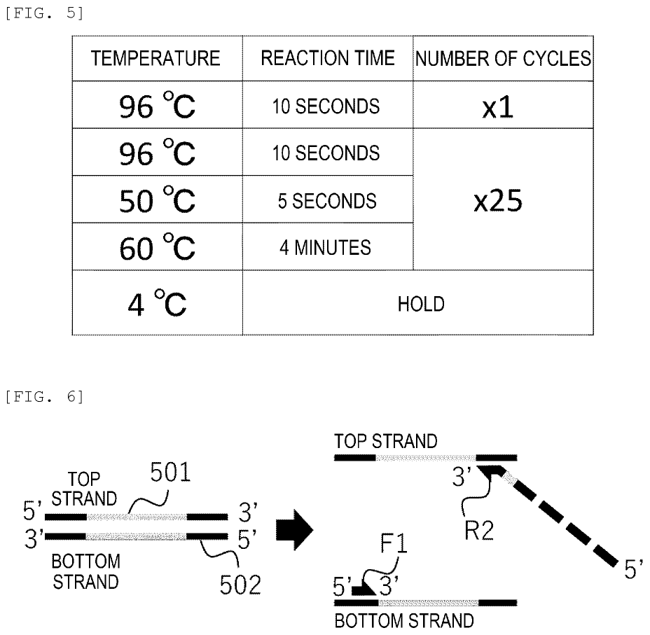

Next, effects of the embodiment were confirmed by using actual samples. FIG. 3 is a diagram showing information on a primer, FIG. 4 is a flowchart showing an effect confirmation procedure, and FIG. 5 is a diagram showing temperature conditions in the cycle sequence reaction. Hereinafter, Example and Comparative Examples will be separately described.

### EXAMPLE

First, Example will be described. FIG. 6 is a conceptual diagram of a DNA and primers used in Example.

As the target polynucleotide, a PCR fragment 502 obtained by amplifying 5 ng of a pUC18 DNA with a primer F1 (corresponding to the first primer 203) and a primer R1 shown in FIG. 3 was used (step S401). Next, the PCR fragment was purified with a DNA purification column (step S402) and diluted with a TE buffer to 1 ng/μl. Thereafter, 1 μl (4 pmol) of the primer F1 and 1 μl (4 pmol) of a primer R2 (corresponding to the second primer 204), 2 μl of a sequence buffer attached to a BigDye™ terminator v3.1 cycle sequencing kit (Applied Biosystems™), 4 μl of BigDye™ terminator 3.1 reaction mix, and 11 μl of MilliQ water were added to 1 μl (1 ng) of the PCR fragment as the target polynucleotide, followed by mixing in a reaction tube having a volume of 0.2 ml. At this time, as shown in FIG. 6, a 3′ side of a DNA of each of the primer F1 and the primer R2 faces an analysis object 501. Further, the reaction tube was loaded into a thermal cycler, and the cycle sequence reaction including the denaturing step, the annealing step, and the extending step was performed under conditions shown in FIG. 5 (step S403).

Next, the reaction product was purified by ethanol precipitation (step S404). In the ethanol precipitation, 2 μl of 125 mM EDTA-2Na (pH 8.0), 2 μl of 3M sodium acetate (pH 5.0), 1 μl (100 ng) of pUC18 DNA for coprecipitation, and 50 μl of 99.5% ethanol were added to the reaction tube and stirred. The pUC18 DNA for coprecipitation is not fluorescently labeled and is thus not detected during the electrophoresis. The reaction tube was allowed to stand at room temperature for 15 minutes to aggregate the DNA, and then centrifuged at 4° C. and 2000 g for 45 minutes. After the centrifugation, the supernatant was discarded, and 70 μl of 70% ethanol was added, followed by centrifugation at 4° C. and 2000 g for 15 minutes. After the centrifugation, the supernatant was discarded, and a precipitated pellet-shaped DNA was air-dried. The DNA pellets were dissolved in 10 μl of high-purity formamide. The obtained DNA solution was subjected to capillary electrophoresis (step S405), and the nucleotide sequence was determined (step S406).

### Comparative Examples

Comparative Examples will be described. In Comparative Examples, a primer added during the cycle sequence reaction in step S403 is different from that in Examples. Specifically, a sample to which only the primer F1 was added was prepared in Comparative Example 1, a sample to which only the primer R1 was added was prepared in Comparative Example 2, and a sample to which the primer F1 and the primer R1 were added was prepared in Comparative Example 3. The primer R1 does not have a reaction stop site and a mobility reduction site. In Comparative Examples, a cycle sequence reaction, purification, and electrophoresis were performed under the same conditions as in Example except for the primer.

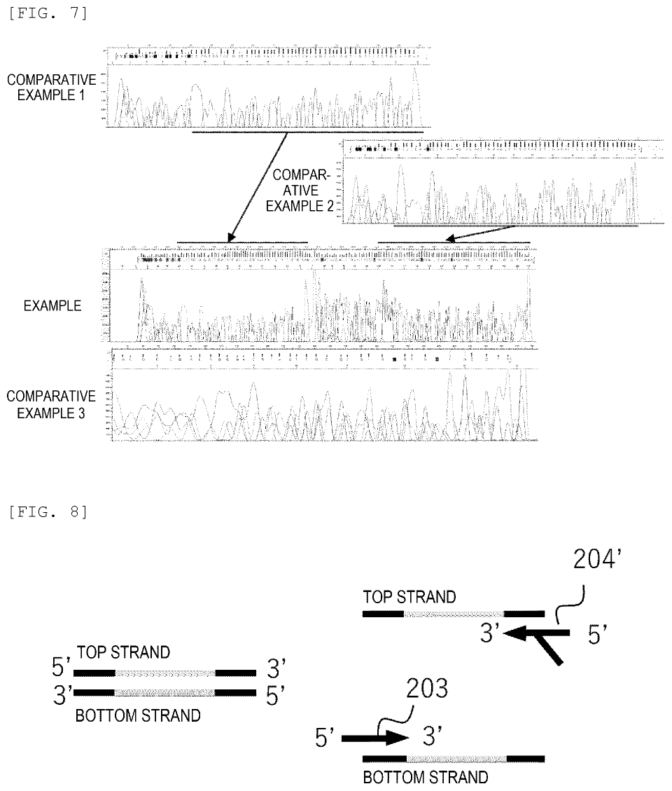

FIG. 7 shows electropherograms obtained as a result of the electrophoresis for Example and Comparative Examples each having a horizontal axis being the number of scans (time) and a vertical axis being a signal intensity. As is clear from FIG. 7, sequence information on only the bottom strand was obtained in Comparative Example 1, and sequence information on only the top strand was obtained in Comparative Example 2. In Comparative Example 3, peaks of both the top strand and the bottom strand are detected, but the peaks cannot be distinguished from each other because the peaks overlap with each other. In contrast, in Example, since peaks and sequence information of both the top strand and the bottom strand are developed on the horizontal axis, the peaks and the sequence information can be clearly determined.

### Embodiment 2

In Embodiment 2, ddNTPs labeled with four different types of fluorescent substances are used for respective one of the four types of bases, and in Embodiment 2, a primer is fluorescently labeled. In the present embodiment, since only one type of substance is used in the fluorescent labeling, cost required for the fluorescent labeling can be reduced. Further, according to the present embodiment, since it is sufficient to read light having one type of wavelength, there is an advantage that a reading device having low performance can also be applied. Wavelengths of fluorescent dyes for labeling the first primer 203 and the second primer 204 may be the same as or different from each other.

In Embodiment 2, a target polynucleotide is dispensed into four reaction tubes during a cycle sequence reaction. Here, the tubes are referred to as a first tube, a second tube, a third tube, and a fourth tube, respectively. A dNTP, the first primer 203, the second primer 204, a thermostable DNA polymerase, and a buffer solution are added to each tube. Next, a ddATP is added to the first tube, a ddCTP is added to the second tube, a ddGTP is added to the third tube, and a ddTTP is added to the fourth tube. Thereafter, steps from a temperature cycle to purification are the same as those in Embodiment 1.

A purified reaction product is separated and detected by electrophoresis. At this time, a sample in each tube is subjected to the electrophoresis in a separate flow path. A DNA fragment group is separated by a molecular screening effect of a separation medium, a fluorescence signal from each primer is detected, and a base sequence is determined based on the detected signal.

Also in the present embodiment, a moving speed of a first fragment group is generally larger than that of a second fragment group. Therefore, a signal derived from the first fragment group is generally detected earlier than a signal derived from the second fragment group, and can be distinguished from each other. Further, in the present embodiment, a dye primer method that is a modification of the Sanger method can be used.

### Embodiment 3

In Embodiment 2, the primer is labeled with a fluorescent dye, and in Embodiment 3, a primer is labeled with a radioactive isotope. For example, an isotope ³²P of phosphorus is used to label the primer, and other radioactive isotopes may be used as long as the radioactive isotopes can be detected. In the present embodiment, an electrophoresis device including no laser or fluorescence detection unit can be used.

### Embodiment 4

In Embodiment 1, a straight-chain-shaped molecule is used as the mobility reduction site in the second primer, and in Embodiment 4, a mobility reduction site in a second primer has a branched structure. FIG. 8 is a conceptual diagram of a DNA and primers according to Embodiment 4. As shown in FIG. 8, a compound having a branched structure is bonded to a 5′ end of a second primer 204′, and a part of a molecule of the second primer 204′ is branched. Examples of the compound having a branched structure include use of an oligodendrimer, a branched polyethylene glycol, and a poly(ADP)adenyl group.

According to the present embodiment, since a mobility decreases in a portion of the branched structure, an effect of reducing the mobility is larger than that in a case of a molecule having a straight chain structure with the same length. Therefore, if a degree of the mobility reduction effect by the branched structure can be predicted, a length of the second primer can be reduced by the degree, which leads to cost reduction. In addition, even when an amplification region has a large strand length, it is possible to prevent the mobility reduction site from being excessively long.

The invention is not limited to the above-described embodiments, and includes various modifications. For example, a part of a configuration of a certain embodiment can be replaced with a configuration of another embodiment, and a configuration of another embodiment can be added to a configuration according to a certain embodiment. In addition, another configuration can be added to, deleted from, or replaced with a part of a configuration of each embodiment.

### REFERENCE SIGNS LIST

201, 501: analysis object
202: target polynucleotide
203: first primer
204, 204′: second primer
205: amplification region
206: signal derived from first fragment group
207: signal derived from second fragment group
502: PCR fragment
2030, 2040: target recognition site
2041: reaction stop site
2042: mobility reduction site

1. A nucleotide sequence identification method for identifying one or more nucleotide sequences in a target polynucleotide complementary pair constituting a double strand, the nucleotide sequence identification method comprising:
a step of identifying, by using a first primer having a target recognition site configured to form a complementary pair by hydrogen bonding with a part of one target polynucleotide of the target polynucleotide complementary pair, one or more nucleotide sequences in the one target polynucleotide; and
a step of identifying, by using a second primer having a target recognition site configured to form a complementary pair by hydrogen bonding with a part of the other target polynucleotide of the target polynucleotide complementary pair and a reaction stop site configured to stop a nucleotide extension reaction by a DNA polymerase, one or more nucleotide sequences in the other target polynucleotide, wherein
a mobility of a second reaction product obtained by the second primer extending a complementary strand on the other target polynucleotide is smaller than a mobility of a first reaction product obtained by the first primer extending a complementary strand on the one target polynucleotide.

2. The nucleotide sequence identification method according to claim 1, wherein
the second primer is longer than the first primer.

3. The nucleotide sequence identification method according to claim 1, wherein
the second primer has a mobility reduction site configured to reduce a moving speed of a reaction product.

4. The nucleotide sequence identification method according to claim 3, wherein
the mobility reduction site is longer than an amplification region sandwiched between the first primer and the second primer.

5. The nucleotide sequence identification method according to claim 1, wherein
the first reaction product and the second reaction product are separated by electrophoresis and detected by fluorescence, and
a ddNTP contained during the extension reaction is labeled with a fluorescent substance.

6. The nucleotide sequence identification method according to claim 1, wherein
the first reaction product and the second reaction product are separated by electrophoresis and detected by fluorescence, and
the first primer and the second primer are labeled with a fluorescent substance.

7. The nucleotide sequence identification method according to claim 1, wherein
the first reaction product and the second reaction product are separated and detected by electrophoresis, and
the first primer and the second primer are labeled with a radioactive isotope.

8. The nucleotide sequence identification method according to claim 3, wherein
the mobility reduction site has a branched structure.
